(19) United States
(12) Patent Application Publication
(10) Pub. No.: US 2025/0292441 A1
(43) Pub. Date: Sep. 18, 2025

(54) Method and System for Compressing Medical Image Data

(71) Applicant: Siemens Healthineers AG, Forchheim (DE)

(72) Inventor: Mario Zeller, Erlangen (DE)

(73) Assignee: Siemens Healthineers AG, Forchheim (DE)

(21) Appl. No.: 19/081,225

(22) Filed: Mar. 17, 2025

(30) Foreign Application Priority Data
Mar. 18, 2024 (EP) 24164204.0

(51) Int. Cl. G06T 9/00; G06N 3/0455; G16H 30/40

(52) U.S. Cl. CPC G06T 9/002; G06N 3/0455; G16H 30/40

(57) ABSTRACT

A computer-implemented method may include receiving, at an input layer of a trained autoencoder, the digital medical image data set from the medical scanner. The trained autoencoder may include at least one extra dimension in the input layer and optionally in an output layer. The at least one extra dimension is provided in particular in response to a received notification of at least one data redundancy dimension of the received digital medical image data set. The method may include compressing, by the trained autoencoder, at least one part of the received digital medical image data set using the received notification of the at least one data redundancy dimension and/or data received by the at least one extra dimension.

### CROSS REFERENCE TO RELATED APPLICATIONS

This patent application claims priority to European Patent Application No. 24164204.0, filed Mar. 18, 2024, which is incorporated herein by reference in its entirety.

### BACKGROUND

### Field

A technique is provided for compressing a digital medical image data set, which was obtained using a medical scanner. The technique may comprise a method, a device, a system, a computer program product, and a computer-readable medium.

### Related Art

An autoencoder is an artificial neural network, which is used to learn efficient encoding. The aim of an autoencoder is to learn a compressed representation (also: encoding) for a set of data and thus also to extract essential characteristics. Accordingly, the autoencoder can be used for dimension reduction. Autoencoder architectures can be used, e.g., in image or video compression, as described by Amirhossein Habibian et al. in “Video Compression With Rate-Distortion Autoencoders”, arXiv: 1908.05717 [eess.IV], the content of which is incorporated by reference herein.

A simple autoencoder-decoder architecture is reproduced in FIG. 4 in accordance with Fig. (D) by Christ Kuo/Dr. Dataman, “Convolutional Autoencoders for Image Noise Reduction”, Towards Data Science, 20 Nov. 2019, the content of which is incorporated by reference herein.

The left part 402 of the autoencoder-decoder in FIG. 4 is designated the encoder, the right part 404 the decoder. In the example case shown in FIG. 4, an input image of 28×28 pixels is used (or read) at the input layer 406 of the encoder 402. Multiple folding layers 408-1; 408-2; 408-3 then follow, which each have a smaller matrix size but increased number of channels. The smallest matrix is then transferred or flattened to a one-dimensional (1D) vector at reference sign 410. FIG. 4 does not show rectified linear unit (ReLU) layers between the individual folding layers 408-1; 408-2; 408-3. The size h at reference sign 412 is designated a compressed representation, compressed image, code, or latent. In the case shown in FIG. 4, the compressed image 412 only comprises ten (10) values. The compressed image 412 is required later to obtain the final image at the output layer 420 of the decoder 404 on the right-hand side of FIG. 4. To achieve this, the process is now effected in reverse. Vector 414 is transformed at reference sign 416 into a smallest matrix size. This is followed by unfolding layers 418-1; 418-2 and the outputting of the decompressed image or final image to the output layer 420 of the decoder 404. The autoencoder-decoder can later be divided into encoder 402 and decoder 404, wherein the decoder 404 only needs the code 412 of the respective input image later on, in order to restore the output image.

For image compression tasks, network training includes presenting the image that is as identical as possible for the input and output image in each case and using, e.g., the mean squared error (MSE) as the loss function between the achieved output image and input image. For two-dimensional (2D) digital camera images, it is known that a raw data volume, which represents a high-resolution image with a large number of sensors or pixels, contains redundancies and can be compressed without significant information loss. It is also possible to scan only a small part of the information by means of sensor data, in an extreme case with a one-pixel camera, and then to use the sensor data to compile an image mathematically. This observation makes use of, e.g., compressed sensing.

Medical, in particular three-dimensional (3D) image data (e.g., magnetic resonance imaging (MRI) scans) usually has a certain amount of data redundancy. This can arise in the time dimension in time-series scans, such as functional (fMRI) or dynamic imaging, in the contrast dimension in multi-contrast scans, such as Dixon imaging or mapping scans, in the layer dimension, or in the coil dimension in multi-channel scans.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a flowchart of a method for compressing a digital medical image data set, which was obtained using a medical scanner, according to an exemplary embodiment of the present disclosure.

FIG. 2 is a flowchart of a training phase of an autoencoder according to an exemplary embodiment of the present disclosure, in order to later execute, in an inference phase, the method for compressing a digital medical image data set, which was obtained using a medical scanner, in accordance with FIG. 1.

FIG. 3 illustrates an autoencoder network according to an exemplary embodiment of the present disclosure, where the autoencoder network may execute the method of FIG. 1 in accordance with the training phase of FIG. 2.

FIG. 4 illustrates a conventional folding autoencoder-decoder network for conventional 2D image compression.

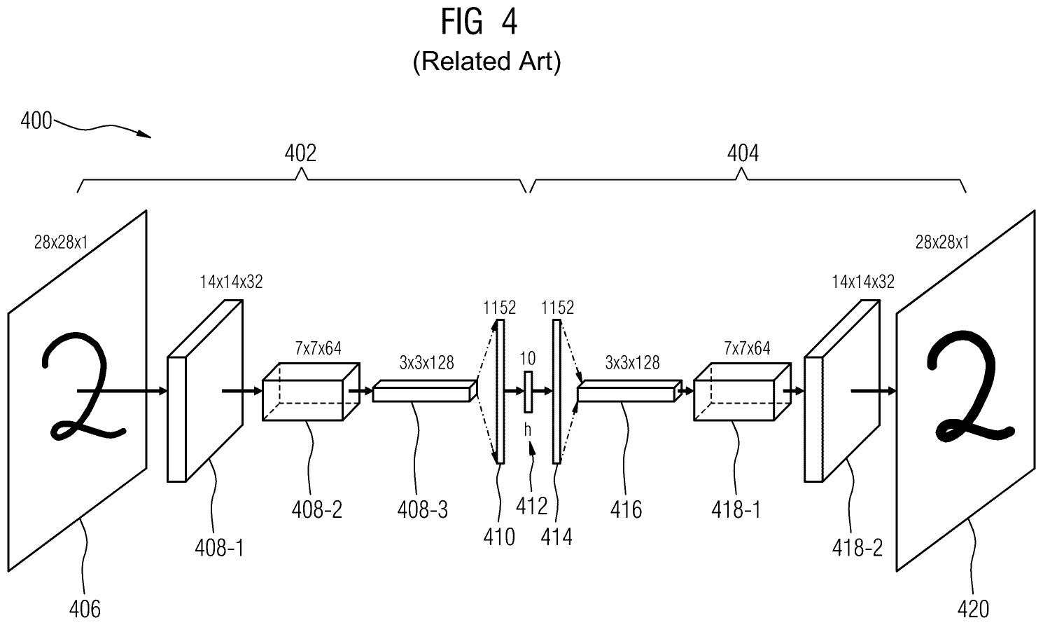

FIGS. 5A-5D illustrate examples of a head scan with Haar wavelet transformations according to exemplary embodiments of the present disclosure.

The exemplary embodiments of the present disclosure will be described with reference to the accompanying drawings. Elements, features and components that are identical, functionally identical and have the same effect are-insofar as is not stated otherwise-respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the disclosure is to provide a solution for compressing digital medical image data sets. Alternatively, or additionally, the object is to provide a technique for saving storage space and/or resources when transmitting digital medical image data.

The disclosure includes a method for compressing a digital medical image data set, which was obtained using a medical scanner, by an autoencoder network, by a system, by a computer program or computer program product, and by a computer-readable storage medium in accordance with the attached independent claims. Advantageous aspects, features, and embodiments, as well as advantages, are described in the following description.

The solution according to the disclosure is described below in reference to a method for compressing a digital medical image data set, which was obtained using a medical scanner, and in reference to the claimed autoencoder network (for short: autoencoder). Features, advantages, or alternative embodiments herein can be assigned to the other aspects (e.g., the system comprising the autoencoder network, the computer program, or a computer program product) and vice versa. In other words: aspects of the autoencoder network and/or the system comprising the autoencoder network can be improved by features that are described in connection with the method. In this instance, the functional features of the method are embodied by structural entities of the autoencoder network and/or the system and vice versa.

In accordance with an aspect of the method, an (in particular computer-implemented) method is provided for compressing a digital medical image data set, which was obtained using a medical scanner. The method may comprise the step of receiving a notification of at least one data redundancy dimension with regard to a digital medical image data set to be received. The method can further comprise a step of selecting a trained autoencoder in response to the received notification.

The method may further comprise the step of receiving the digital medical image data set from the medical scanner at an input layer of the (or a) trained autoencoder. The trained autoencoder can comprise at least one extra dimension in the input layer. The trained autoencoder may also optionally comprise at least one extra dimension in an output layer. The at least one extra dimension (in particular in the input layer) can be designed to receive a notification of at least one data redundancy dimension of the received digital medical image data set. Alternatively, or additionally, the extra dimension (in the input layer and/or optionally in the output layer) can be provided in response to a received notification of at least one data redundancy dimension of the received digital medical image data set. Furthermore, the at least one extra dimension in the input layer can alternatively or additionally be designed to receive data with regard to the data redundancy dimension. The data received at the at least one extra dimension in the input layer can comprise at least one part of the received digital medical image data set, selected in particular with regard to the data redundancy dimension.

The notification of the at least one data redundancy dimension of the received digital medical image data set can be implicit and/or be received implicitly (e.g., the trained autoencoder can be selected and/or controlled for a selected data redundancy dimension). In this context, “be implicit” or “be received implicitly” means being implicitly received with the other data received in the input layer. For example, the notification can be derived from the received digital medical image data set or a part of the same and thus the notification is already implicitly included in the received data. Alternatively, or additionally, it is possible for a user to input the notification via a user interface or select it via a field on the user interface.

The method may further comprise the step of compressing, by means of the trained autoencoder, at least one part of the received digital medical image data set using the received notification of the at least one data redundancy dimension and/or using data received by the at least one extra dimension (e.g., comprising at least one partial copy of the digital medical image data set, in particular comprising adjacent layers and/or chronological instances of a multi-layer and/or time-series scan, for example, for a layer dimension or a time dimension as a data redundancy dimension).

Less storage space is required for (in particular conventionally extensive) digital medical image data due to the technology according to the disclosure for compressing a digital medical image data set. Alternatively, or additionally, the technology according to the disclosure for compressing a digital medical image data set can facilitate a resource-saving, prompt, complete, and/or error-free transmission of digital image data, in particular with low transmission bandwidth and/or low transmission speed.

The medical scanner (for short: scanner) can also be designated a medical imaging scanner and/or a magnetic imaging system. Alternatively, or additionally, the medical scanner can comprise a magnetic resonance imager (MRI), a computer tomograph (CT), a positron emission tomograph (PET), a single photon emission computed tomograph (SPECT), or a combination thereof (e.g., PET/CT or SPECT/CT).

The MRI can also be called an MRI scanner.

In one exemplary embodiment, the trained autoencoder suitable for the received notification of the at least one data redundancy dimension can be selected, for example, by an upstream and/or higher authority. The upstream and/or higher authority can be a digital processing unit and select the suitable autoencoder, which is extremely well suited to processing the received digital medical image data set by exploiting a data redundancy (e.g., in a specific direction). In MRI imaging (at least a certain) data redundancy is usual, especially in the coil dimension (more and more coil channels are scanned now in particular), which is why an autoencoder optimized for the coil dimension can lead to a data reduction for all MRI image data sets, even if a specific autoencoder can deliver even better results for certain applications (e.g., data redundancy in the time direction).

The at least one extra dimension in the input layer can be at least one additional neuron, which is provided in particular in addition to the neurons of the input layer of a conventional autoencoder.

Alternatively, or additionally, the input layer can be subdivided into at least two sublayers. For example, a first sublayer (also: input interface) can be designed for (e.g., conventional) receiving of the digital medical image data set (and/or comprise the conventional input neurons of the autoencoder). A second sublayer can comprise the at least one extra dimension (and/or the additional neurons). For example, the second sublayer can rearrange, duplicate, and/or bundle parts (e.g., layers and/or chronological instances of a time-series scan) of the digital medical image data set. The processing of the digital medical image data set using the data redundancy dimension can then be effected, in particular based on rearranging, duplicating, and/or bundling in the second sublayer, in subsequent hidden layers of the autoencoder.

The data redundancy dimension (for short: redundancy dimension) can be (in particular at least) one other dimension of the input layer of the trained autoencoder, via which the redundant data is captured. The data redundancy (for short: redundancy) can-depending on the application-be based on different technical parameters or aspects. This allows the data redundancy to be, e.g., specific for a type of scan, e.g., in terms of the modality of the medical scanner, of a scanning protocol (also: imaging protocol) and/or an underlying medical issue. Alternatively, or additionally, the data redundancy, or thus the data redundancy dimension, can comprise, in particular, a time dimension (e.g. for a scan across several cardiac cycles and/or respiratory cycles), a layer dimension (e.g., for a 3D scan made up of layers), a contrast dimension (e.g., for a multi-contrast scan), and/or a coil dimension (e.g., for a multi-channel MRI scan). For example, the data redundancy or the data redundancy dimension can be assigned to an imaging protocol (e.g., comprising multi-channel coils) and/or at least one acquisition parameter. Alternatively, or additionally, the data redundancy or the data redundancy dimension can be caused by overlapping imaging (in particular in overlapping anatomical areas), by multiple scans or repeated measurements, by using multi-coil systems, and/or by parallel measurements.

The notification of the at least one data redundancy dimension can be calculated from the received digital medical image data set. Alternatively, or additionally, the notification can be calculated from metadata. The metadata can comprise the type of medical issue, the modality, the at least one acquisition parameter, data relating to the scanning protocol, and/or data relating to the scanner. Alternatively, or additionally, the notification can be implicit to at least one data redundancy dimension. E.g., the autoencoder can deduce or derive the data redundancy dimension from the received digital medical image data set and/or from the metadata.

The digital medical image data set (hereinafter for short also: medical image data set) can comprise raw data that was obtained, e.g., by means of an MRI scan, in particular k-space data. Alternatively, or additionally, the medical image data set can comprise pre-processed and/or (e.g., Fourier-) transformed data, which is based on raw data. Alternatively, or additionally, the medical image data set can comprise reconstructed images. The medical image data set can be obtained directly from an MRI scanner. Alternatively, the medical image data set can be read from a memory.

The trained autoencoder can comprise an encoder of a trained autoencoder-decoder. A network architecture of the encoder and decoder can be independent of each other. Alternatively, or additionally, in one exemplary embodiment the decoder can comprise an inverse layer architecture of the encoder.

An autoencoder (for short: encoder) can comprise an input layer. The input layer can be designed to receive a (or the) digital medical image data set, which was scanned by the medical scanner (e.g., MRI), (or a pre-processed and/or transformed version of the digital medical image data set).

The at least one extra dimension in the input layer can be designed to exploit the data redundancy (in particular in the data redundancy dimension). With a scan of 64 repetitions with, e.g., 256×256 matrices, each of the 256×256 matrices are not, for example, successively fed into (and/or sent to) the autoencoder, but rather a block of 256×256×64. Thus, the repetition dimension represents the extra dimension in this example.

The autoencoder can also comprise a plurality of folding layers (in technical terminology: convolutional layers). Alternatively, or additionally, the autoencoder can comprise one or more rectified linear units (ReLU) and/or max pooling layers.

The autoencoder can also comprise an (e.g., output) layer (e.g., a fully connected layer), which provides a compressed representation (also: compressed version, code, or latent) of the medical image data set.

The term “compression” should be understood in the sense of producing an efficient representation of the digital medical image data set. The production of an efficient representation can mean a pure data compression. Alternatively, or additionally, the production of an efficient representation can comprise a data reconstruction and/or dimensionality reduction.

The compression can also be designated encoding. The result of the compression can be a compressed version (also: representation) of the medical image data.

The compression can be lossy. The compression can, e.g., comprise storage of a fraction (e.g., a tenth of the storage space) of the (in particular originally) received digital medical image data set (e.g., 800 MB after compression and/or encoding instead of 8 GB before compression and/or encoding).

The compressed version of the medical image data set can comprise the compression of at least one part of the received digital medical image data set. For example, the at least one part of the received digital medical image data set can comprise just one part of the k-space of an MRI scan, for which compression was performed.

The compressed version of the medical image data set can be provided to an autodecoder (for short: decoder) (e.g., to an input layer, in particular to a fully connected layer).

The autodecoder can comprise a plurality of unfolding layers (in technical terminology: deconvolutional layers). Alternatively, or additionally, the autodecoder can comprise one or more ReLUs and/or max depooling layers.

The autodecoder can also comprise an output layer. The output layer can be designed to provide a decompressed digital medical image data set. The decompressed digital medical image data set can comprise a decompression of the compressed version of the medical image data set.

The decompression and the original medical image data set may match in key characteristics (e.g., in terms of type and position of anatomical structures and/or in terms of presence, position, and size of an abnormality, in particular a lesion and/or tumor).

The autoencoder and the autodecoder can be trained together, in order to maximize a similarity between the (in particular originally) received digital medical image data set and the decompressed medical image data set. For example, a loss function can comprise a mean squared error (MSE; and/or-possibly until overall normalization-equivalent to the L2 loss), a mean absolute error (MAE; and/or-possibly until overall normalization-equivalent to the L1 loss) and/or a structural similarity index measure (SSIM). The MSE, MAE, and/or SSIM can be determined per voxel (and/or per pixel). Alternatively, or additionally, the loss function can comprise a mean of the MSE, MAE, and/or SSIM per voxel (and/or per pixel).

The autoencoder (and optionally the autodecoder) can be trained for different types and/or realize in different exemplary embodiments the at least one extra dimension (in particular in the input layer and optionally in the output layer). The different exemplary embodiments and/or training types can be combinable with each other.

In accordance with a first exemplary embodiment, the autoencoder (and optionally the autodecoder) can be trained for one or more maximum potential data redundancy dimensions. For a layer dimension the maximum number can be, e.g., 256. If there are fewer layers to compress, e.g., just 30, individual layers that include zeros can, e.g., be added (in particular artificially).

In a second exemplary embodiment, the autoencoder (and optionally the autodecoder) can be trained for a predetermined (and/or “typical”) number of layers, e.g., 30 layers. If there are then, e.g., 60 layers in the received digital medical image data set, the input data set can be cut appropriately, in the simplest case into two blocks of 30 layers each.

In a third exemplary embodiment, in a (particularly more complex) case, the blocks of the autoencoder (and optionally of the autodecoder) can have a partial (and/or certain) overlapping (e.g., “sliding window”). This makes it possible to avoid the formation of visible transitions (in particular at the edges, e.g., of the digital medical image data set). E.g., an overlap of three (3) layers can be formed.

In a fourth exemplary embodiment, in order to learn more information about the data redundancies, an adjacent matrix can also be transmitted in the input layer (e.g., the two adjacent layers of the layer to be compressed). This makes it possible to save resources, as a smaller compression of the digital medical image data set (also: the latent vector) can be selected, because more data redundancies can be detected or because, with the same number of output data sets, an averaging of the same matrix transmitted at different positions in multiple transmission processes can be effected.

The method can further comprise a step for transmitting the compressed digital medical image data set. Alternatively, or additionally, the method can further comprise a step for storing the compressed digital medical image data set.

The compressed digital medical image data set can be transmitted to a cloud, a computing device (and/or an outsourced computing cluster) physically separate from the medical scanner (e.g., MRI), and/or a (particularly central) memory (e.g., of a radiology department, a medical facility, and/or a database for electronic patient files, in technical terminology: electronic health records, EHRs). For example, an image reconstruction can be executed through a cloud-based process.

The transmission of the compressed digital medical image data set can be resource-saving (e.g., with regard to bandwidth and/or time) and/or reliable, e.g., with a slow (in particular wired and/or wireless) internet connection.

Alternatively, or additionally, the compressed digital medical image data set can be saved (e.g., after transmission), e.g., in the (particularly central) storage (and/or in the cloud). The compression saves a great deal (e.g., by a factor of ten) of space in comparison to the conventional storage space. This enables optimized use of storage capacities.

The received notification of the at least one data redundancy dimension can be information (e.g., comprising one or more bits) with regard to a type of data redundancy. Alternatively, or additionally, the received notification can be a selection (and/or an activation command), in particular with regard to the at least one extra dimension, of the trained autoencoder from a number of trained autoencoders, wherein each autoencoder from the number of trained autoencoders is trained for a predetermined (in particular different) type of data redundancy. Alternatively, or additionally, the received notification can be a copy of at least one part of the digital medical image data set (that is received in particular by the conventional part of the input layer).

The received notification of the at least one data redundancy dimension (for short: notification) can show which data dimension is probably redundant (e.g., coil channels, time, 2D layers, and/or anatomical region). The notification can in particular be calculated algorithmically or derived from metadata (e.g., from the image data, from the modality, from the imaging protocol, and/or from a medical issue that is the reason for the imaging).

In one embodiment variant, the notification can be captured via the at least one extra dimension (for short: the extra dimension) of the autoencoder. Thus, in this embodiment variant the extra dimension is used to capture the notification. The compression can be tuned and/or turned on or off accordingly. In particular, a comprehensive model (and/or an autoencoder with a multitude of, in particular hidden, layers and/or neurons) can be trained for different compression scenarios.

In another embodiment variant, the notification can define the architecture of the autoencoder. In this embodiment variant, the notification is only used to select the suitable (and/or suitably trained) autoencoder from a database of multiple autoencoders, which are trained for different applications. A “small, specific” autoencoder, e.g., for cardiac MRI (in particular with data redundancy in the time dimension) and/or for multi-contrast MRI (in particular with data redundancy in the contrast dimension) can be saved in the database in trained form. The “small, specific” autoencoder is only selected in the inference. In this embodiment variant, various small autoencoders can be modular and easier to train in each instance.

The notification or the data captured in the extra dimension (in particular in the input layer) can comprise (in particular resource-saving) information (e.g., “multi-contrast MRI”, in particular expressed by a short bit sequence) or the (e.g., multi-contrast MRI) image data itself.

The data redundancy along the data redundancy dimension can be exploited because, e.g., in a cardiac time series mainly the image information in the vicinity of the heart changes, while the image information at the edge essentially remains static.

The at least one extra dimension (for short: the extra dimension) in the output layer can be designed to provide information regarding the received notification that has been used in the compression step to an autodecoder (in particular trained together with the autoencoder). For example, the compressed digital medical image data set can comprise one or more bits (in particular at a predetermined place in a bit sequence), which encode the type of data redundancy dimension. The one or more bits that encode the type of data redundancy dimension can be concatenated with the compressed digital medical image data set.

The extra dimension in the output layer of the autoencoder is not required in some embodiment variants. E.g., with multi-layer scans, information from adjacent layers can be used to achieve better compressibility. Areas in which similar textures or just noise also occur in adjacent layers can then be compressed more heavily. An autodecoder trained together with the autoencoder can accept the necessary information about the data redundancy dimension as implicitly fulfilled when it receives the compressed digital medical image data set.

The method can further comprise a step for determining a compression factor for compressing at least one part of the received digital medical image data set.

The compression factor can be adjusted for a flexible network architecture of the autoencoder (and/or the autodecoder). The autoencoder and the autodecoder can each be trained for the same compression factor and decompression factor. The flexibility of determining (also: defining) the compression factor can advantageously be dependent on a type of the medical (e.g., MRI) scan. In one exemplary embodiment, an autoencoder can, for example, be trained for a multitude of MRI scans in accordance with different imaging protocols.

The medical scanner can comprise MRI, CT, PET, and/or SPECT as the modality. In particular, two or more modalities can be combined, e.g., for molecular imaging.

The combination of two or more medical scanners (and/or modalities), in particular for molecular imaging, can comprise PET and CT (for short: PET/CT) in one exemplary embodiment. In another exemplary embodiment, the combination of two or more medical scanners (and/or modalities), in particular for molecular imaging, can comprise SPECT and CT (for short: SPECT/CT).

The technology according to the disclosure for compressing a digital medical image data set can be applied to image data for each medical imaging modality.

The digital medical image data set can be two-dimensional (2D) or three-dimensional (3D, also: volumetric). For example, a 2D digital medical image data set can comprise one layer. Alternatively, or additionally, a 3D digital medical image data set can comprise a stack of layers.

The received digital medical image data set (for short: medical image data set) can comprise raw data of a scan from the medical scanner. Alternatively, or additionally, the medical image data set can comprise data in a transformed space. Alternatively, or additionally, the medical image data set can comprise data in the k-space of an MRI scan. Alternatively, or additionally, the medical image data set can comprise data in the (in particular complex) image space of a scan from the medical scanner. Alternatively, or additionally, the medical image data set can also comprise data in a hybrid space of an MRI scan.

The data in the transformed space can be transformed using a Fourier transformation (FT), wavelet transformation (WT), and/or sparsity transformation (ST).

The FT can, for example, comprise a fast Fourier transformation (FFT).

The WT (wavelet transformation) can comprise a linear time-frequency transformation with a wavelet analysis, which indicates the transition of the time representation to the spectral or wavelet representation, and a wavelet synthesis, which indicates the back transformation of the wavelet-transformed data to the time representation. Alternatively, or additionally, the WT can be a continuation of the short-term Fourier transformation (STFT).

By means of (e.g., 2D discrete) Haar WT, spectral information from the medical image data set (e.g., of a head image) can be exploited, for example. The Haar WT can correspond to a discrete cosine transformation, which is applied one after the other in the horizontal and vertical direction in an image-pixel rectangle.

The ST can produce a sparsity of data in any dimension of the input data set. Known STs are Sobel, Scharr, or Laplace filters, singular value decomposition, or total variation approaches. In particular, the ST can also comprise an FT or WT, however.

The data in the hybrid space can be Fourier transformed in a one-dimensional (1D) time direction and/or read direction (also: phase encoding direction in the k-space). For example, an FT of a cardiac MRI time series can be executed in the time direction. Only Fourier coefficients with low frequency are found in places with little changes in the image; the remainder (in particular Fourier coefficients with high frequencies) is approximately zero.

The different data types can correspond to different stages of data processing of the digital medical image data set. Alternatively, or additionally, the technology according to the disclosure for compressing a digital medical image data set can be applied at different stages of the processing of the digital medical image data set.

The data redundancy dimension can comprise a time dimension, if the digital medical image data set includes a time-series scan. Alternatively, or additionally, the data redundancy dimension can comprise a repetition dimension, if the digital medical image data set may comprise a number of repetitions. Alternatively, or additionally, the data redundancy dimension can comprise a contrast dimension, if the digital medical image data set includes a multi-contrast scan. Alternatively, or additionally, the data redundancy dimension can comprise a channel dimension, if the digital medical image data set includes a multi-channel scan. Alternatively, or additionally, the data redundancy dimension can comprise a layer dimension, if the digital medical image data set includes a multi-layer scan. Alternatively, or additionally, the data redundancy dimension can comprise a coil dimension, if the digital medical image data set includes a scan with multiple coils.

The received notification of the at least one data redundancy dimension can comprise two or more data redundancy dimensions.

The data redundancy in the time dimension can comprise individual instances (e.g., per time of the scan) including redundant information within the time series. For example, chronologically adjacent instances can comprise a major data redundancy. Alternatively, or additionally, instances at the same time within an (e.g., cardiac and/or respiratory) cycle can have a major data redundancy.

The data redundancy in the repetition dimension can comprise the digital medical image data set including a scan with a multitude of repetitions (e.g., comprising a multitude of respiratory cycles and/or cardiac cycles).

The data redundancy in the contrast dimension can comprise redundant information from scans being identifiable (and/or combinable) with different image contrasts in the digital medical image data set. For example, T1-weighted MRI sequences (T1w), T2-weighted MRI sequences (T2w), T2 *-weighted MRI sequences (T2*w), diffusion-weighted MRI sequences (DWI), and/or dynamic contrast-enhanced MRI sequences (DCE) can be combined with each other. Alternatively, or additionally, Dixon imaging can be used.

Dixon imaging exploits the fact, for example, that water molecules and fat molecules resonate at a different frequency and alternate between in-phase and out-of-phase over time. Water images and fat images can be separated by means of addition and subtraction of the in-phase and out-of-phase measurements.

The data redundancy in the channel dimension can comprise different (e.g., measurement) channels within the digital medical image data set including redundant information.

The data redundancy in the layer dimension can comprise redundant information being included in (in particular adjacent) layers of a volumetric scan that is part of the digital medical image data set.

A sliding window for adjacent instances or layers can be used for exploiting the data redundancy in the time dimension and/or in the layer dimension. Alternatively, or additionally, folds can be performed on adjacent voxels (and/or adjacent pixels), in particular for a data redundancy in the layer dimension and/or the coil dimension.

The data redundancy in the coil dimension can comprise measurements from multiple coils within the digital medical image data set including redundant information. For example, the measurement using one coil can correspond to an MRI scan for one channel. Alternatively, or additionally, a scan with multiple coils can comprise a multi-channel scan.

The redundant information can be compressed by means of the technology according to the disclosure. In particular, the autoencoder can compress the data in a targeted manner along the data redundancy dimension based on the received notification, e.g., based on a-priori knowledge of a type of medical scan included in the notification.

The digital medical image data set can be obtained from the medical scanner in accordance with a predetermined imaging protocol.

The predetermined imaging protocol can comprise a protocol in accordance with the Human Connectome Project (HCP). Alternatively, or additionally, the predetermined imaging protocol can comprise a protocol of functional magnetic resonance imaging (fMRI, also: fMRT). Alternatively, or additionally, the predetermined imaging protocol can comprise a protocol of diffusion tensor imaging (DTI). Alternatively, or additionally, the predetermined imaging protocol can also comprise a protocol of an apparent diffusion coefficient (ADC) scan (e.g., a diffusion-weighted, DWI, MRI sequence).

The HCP can be a series of studies, for example, concerning the human brain.

The fMRI can comprise an imaging method for representing physiological functions inside a patient's body using the techniques of the MRI.

The DTI can comprise an imaging method for measuring the diffusion movement of water molecules in body tissue and depicting their spatial dispersion using the MRI, in particular for examinations of the brain.

When the predetermined imaging protocol is known, the autoencoder can be trained specifically in particular for the compression task using the data redundancy dimension. Alternatively, or additionally, a particularly small autoencoder can be selected (e.g., in terms of a number of layers and/or neurons).

The medical scanner can comprise an MRI. The at least one compressed part of the digital medical image data set can comprise a k-space center, a k-space periphery, and/or alternating different parts of the k-space.

Alternatively, or additionally, the at least one compressed part of the digital medical image data set can comprise an area in the image space, in particular a center and/or a periphery of the image space.

For example, a compression can comprise parts of the image space for cardiac imaging (where there is a particular interest in scans of the beating heart), in order to avoid aliasing artifacts. In particular, it is often necessary to include a patient's thorax. E.g., areas in the image space that comprise parts of the thorax without heart can be compressed differently (in particular more heavily).

The part to be compressed of the digital medical image data set can be selected on the basis of a medical issue and/or a particularly high expected data redundancy.

Advantageously, only the (in particular k-space) center of the image data set can be compressed, e.g., for time series for which high-frequency changes are predominantly expected, e.g., fMRI scans of the head.

Advantageously, only the (in particular k-space) periphery of the image data set can be compressed, e.g., for scans on which the structure remains the same but the basic contrast changes, e.g., for multi-contrast scans and/or in Dixon imaging.

Advantageously, alternating different parts of the image data set (in particular the k-space) can be compressed, e.g., for data that is later reprocessed in an additional reconstruction step and for which a lossy compression can be tolerated well at individual points, as long as it does not always relate to the same spatial sections of the image, e.g., for radial (and/or spiral) or magnetic resonance fingerprinting (MRF) imaging.

Conventionally, the MRI imaging can comprise a Cartesian scan, e.g., line for line in a k-space matrix. Alternatively, or additionally, k-space lines can be scanned on a non-Cartesian basis, e.g., radially or spirally, as described by Pedro F. Ferreira et al. in “Cardiovascular magnetic resonance artefacts”, J Cardiovasc Magn Reson 15, 41 (2013), the content of which is incorporated by reference herein. An analogous principle can be applied to CT imaging, in which the X-ray source and detector are rotated (e.g., in a circular manner) around the patient. MRF imaging is, e.g., a type of quantitative MRI imaging, in which a pseudo-randomized scanning strategy is applied. Clear signal patterns (also: fingerprints) are produced for different materials and/or types of tissue, after which a pattern recognition algorithm maps the fingerprints onto a predetermined lexicon of expected signal patterns.

The technology according to the disclosure can be executed in a very quick and resource-saving manner because only partial compression is involved.

Compression of the at least one part of the received digital medical image data set can also be based on (and/or the received notification of the at least one data redundancy dimension can comprise) a sliding window encoding of adjacent layers within the image data set and/or adjacent chronological instances of a time-series scan within the image data set. Compression based on sliding window encoding can be effected in particular in the sense of compression using the data redundancy dimension and/or also for compression using an additional data redundancy dimension.

With sliding window encoding, a multitude of (e.g., 20) layers can be encoded individually. In order to improve compression, in addition to the layer to be encoded, the (e.g., two) adjacent layers (or for edge layers, e.g., a matrix with zeros) can also be encoded as input data (for short: input) of the autoencoder.

Sliding window encoding can be equivalent to merely saving differences in a layer relative to an (e.g., first) reference layer.

Another aspect of sliding window encoding can comprise the autoencoder learning (or being trained for) a fixed number of layers, but being usable for any number of layers. If, for example, five (5) layers are encoded and step size 1 runs over the 20 input layers, this first increases the number of data packets to be transmitted. This can be prevented by selecting a larger step size (also known as a jumping window). Alternatively, or additionally, data compression can be increased by the existing redundancy between the layers, and thus the increased number of data packets to be transmitted is compensated. Alternatively, or additionally, layers that were transmitted in more than one data packet (e.g., layer 3 can be transmitted for three layers in the extra dimension at position 5, 4, 3, 2, 1 in the respective data packets 1, 2, 3, 4, and 5) are averaged after being received.

Alternatively, or additionally, compression of the at least one part of the received digital medical image data set can also be based on (and/or the received notification of the at least one data redundancy dimension can comprise) a symmetry property of the image data set, in particular a Hermitian k-space symmetry of an MRI scan. Compression based on the Hermitian k-space symmetry can comprise the four quadrants of the k-space containing redundant pieces of information, which can be mapped on top of each other by mirroring and complex conjugation.

Compression based on the symmetry property of the image data set can be effected in particular in the sense of compression using the data redundancy dimension and/or also for compression using an additional data redundancy dimension. For example, the k-space can be viewed as approximately (and/or ideally) point-symmetric to its center (e.g., ideal). If the k-space is divided into four quadrants that can be folded over each other so that the center is positioned at the same corner in each instance, the autoencoder is able to learn spatial correlations and redundancies, which can arise from an (in particular also real) certain similarity of the quadrants.

Alternatively, or additionally, compression of the at least one part of the received digital medical image data set can also be based on an oversampling along a readout direction, in particular for an image data set comprising data in the hybrid space of an MRI scan. Compression based on the oversampling can also be effected in particular for compression using the data redundancy dimension. Oversampling can comprise more data measurements of an MRI signal being performed than are necessary for the resolution of the image display. E.g., the MRI signal can be sampled 512 to 1,024 times per echo (in particular also if the display resolution is usually specified as 256 in the frequency encoding direction). Alternatively, or additionally, a Nyquist sampling rate (also: Nyquist frequency) can be at least twice (e.g., four times) the highest frequency of the signal. As this object is only fulfilled by increasing the digitalization rate of a sampling circuit, it usually causes no time loss and occurs “invisibly”.

Sampling with the Nyquist frequency can be effected in the readout direction.

If there is no oversampling with the Nyquist frequency, misalignment may arise at a high frequency if lower frequencies occur.

With suitable oversampling, areas in which almost no signal occurs can be compressed well.

The oversampling factor can be adjustable.

The autoencoder can be trained for the data redundancy dimension. The training can comprise a step of receiving a notification of at least one data redundancy dimension of a digital medical training image data set (for short: training image data set) at (in particular the at least one extra dimension of) the input layer of an autoencoder. The training image data set can have been obtained using a medical scanner. Alternatively, or additionally, the training image data set can have been produced synthetically.

The autoencoder training can further comprise a step of receiving the digital medical training image data set at the input layer of the autoencoder. In particular, receiving the digital medical training image data set can comprise receiving data with regard to the data redundancy dimension at the at least one extra dimension of the input layer. The data received at the at least one extra dimension in the input layer can comprise at least one part of the received digital medical training image data set, selected in particular with regard to the data redundancy dimension.

The training can further comprise a step of compressing, by means of hidden layers of the autoencoder, at least one part of the received digital medical test image data set. In particular, the compression can be effected using the received notification of the at least one data redundancy dimension of the digital medical training image data set and/or using data received at the at least one extra dimension.

The training can further comprise a step of outputting (and/or transmitting) the result of the compression of the at least one part of the received digital medical test image data set from an output layer of the autoencoder to an input layer of an autodecoder. Outputting (and/or transmitting) the result can comprise a notification of the data redundancy dimension used for the compression sent to the output layer of the autoencoder, which is transmitted to the input layer of the autodecoder, for example, concatenated with the compressed digital medical test image data set.

The training can further comprise a step of decompressing the result of the compression of the at least one part of the received digital medical test image data set by means of hidden layers of the autodecoder, in particular based on the transmitted notification of the data redundancy dimension used for compression. The training can further comprise a step of outputting the result of the decompression, in particular at an output layer of the autodecoder, as well as applying a loss function to the received digital medical training image data set and the outputted result of the decompression.

At least the training steps of compression by the autoencoder, outputting by the autoencoder to the autodecoder, and decompression by the autodecoder can be repeated, in order to optimize the applied loss function.

Training the autoencoder can comprise unsupervised learning.

The autoencoder and the autodecoder can be trained together. For example, when optimizing the applied loss function, weights of neurons of the autoencoder and/or weights of neurons of the autodecoder can be modified.

At least in one inference phase following the training phase, the trained autodecoder can be physically separate from the trained autoencoder. For example, the trained autoencoder can be arranged in physical proximity to one or more medical scanners (e.g., in a radiology department). The trained autodecoder (and/or a number of copies of the trained autodecoder) can be arranged in an (in particular medical) facility that is authorized to receive medical patient data. For example, a copy of the autodecoder can be localized in a practice of a referring physician.

The digital medical training image data set can, for example, comprise an HCP data set (in particular an HCP diffusion data set) and/or an fMRI data set.

The loss function can comprise an MSE, MAE, and/or SSIM.

The training can further comprise a backpropagation and/or a gradient method for training the autoencoder.

A convergence of the training can be improved by means of the backpropagation and/or the gradient method.

The autoencoder can be large-dimensioned and/or have an option for filling out input data with zeros, in particular depending on a type of data redundancy dimension and the digital medical training image data set. The autoencoder can be trained with a plurality of different types of digital medical training image data sets and data redundancy dimensions.

An autoencoder that is trained for a fixed predetermined type of application (e.g., data redundancy reduction in a coil dimension of an MRI scan of a knee joint) can be small-dimensioned.

Small-dimensioned and large-dimensioned can relate to the number of neurons per layer (e.g., input layer, hidden layer, and/or output layer) and/or to a number of (in particular hidden) layers.

An autoencoder that is trained for a plurality of applications (e.g., data redundancy reduction in the time, layer, and/or coil dimension of MRI scans of different body parts) can be large-dimensioned.

Zero-filling input data can be dependent on the application. For example, a predetermined number of neurons of the input layer cannot be activated for a predetermined application.

Zero-filling can comprise, e.g., adding 128 zeros each from left and right for 256 data points, in order to achieve a predetermined (and/or desired) data set size of 512.

In accordance with a device aspect, a trained autoencoder network (for short: autoencoder) is provided for compressing a digital medical image data set, which was obtained using a medical scanner. An input layer of the trained autoencoder network is designed to receive the digital medical image data set. The input layer of the trained autoencoder network can also be designed to receive a notification of at least one data redundancy dimension of the received digital medical image data set. The input layer of the trained autoencoder network can comprise at least one extra dimension in the input layer. The at least one extra dimension can be designed to receive the notification of at least one data redundancy dimension of the received digital medical image data set. Alternatively, or additionally, the at least one extra dimension can be provided in response to a received notification of at least one data redundancy dimension of the received digital medical image data set. Alternatively, or additionally, the at least one extra dimension in the input layer can be designed to receive data with regard to the data redundancy dimension. The data received at the at least one extra dimension in the input layer can comprise at least one part of the received digital medical image data set (selected in particular with regard to the data redundancy dimension).

The trained autoencoder network is designed to compress at least one part of the received digital medical image data set using the notification of the at least one data redundancy dimension and/or using data received at the at least one extra dimension.

An output layer of the trained autoencoder network can optionally comprise at least one extra dimension (e.g., provided in response to a received notification of at least one data redundancy dimension of the received digital medical image data set). The at least one extra dimension provided in the output layer can be designed to comprise and/or provide a notification of the data redundancy dimension used for compression.

The autoencoder network can be designed to execute the method in accordance with the method aspect. Alternatively, or additionally, the autoencoder network can be trained in accordance with the method aspect. Alternatively, or additionally, the autoencoder network can also comprise one or each feature that is disclosed in the context of the method aspect for the autoencoder.

In accordance with a system aspect, a system is provided for compressing a digital medical image data set, which was obtained using a medical scanner. The system may comprise a scanner interface to at least one medical scanner and at least one autoencoder network in accordance with the device aspect.

The system can comprise a plurality of autoencoder networks. For example, one autoencoder network can be trained for each type of application.

In accordance with another aspect, a computer program product is provided with program elements, which trigger an autoencoder network to execute the steps of the method for compressing a digital medical image data set, which was obtained using a medical scanner, and/or for training the autoencoder network in accordance with the method aspect, if the program elements are loaded in a memory of the autoencoder network.

In accordance with another aspect, a computer-readable medium is provided, on which the program elements are saved that can be read and executed by an autoencoder network, in order to execute steps of the method for compressing a digital medical image data set, which was obtained using a medical scanner, and/or for training the autoencoder network in accordance with the method aspect, if the program elements are executed by the autoencoder network.

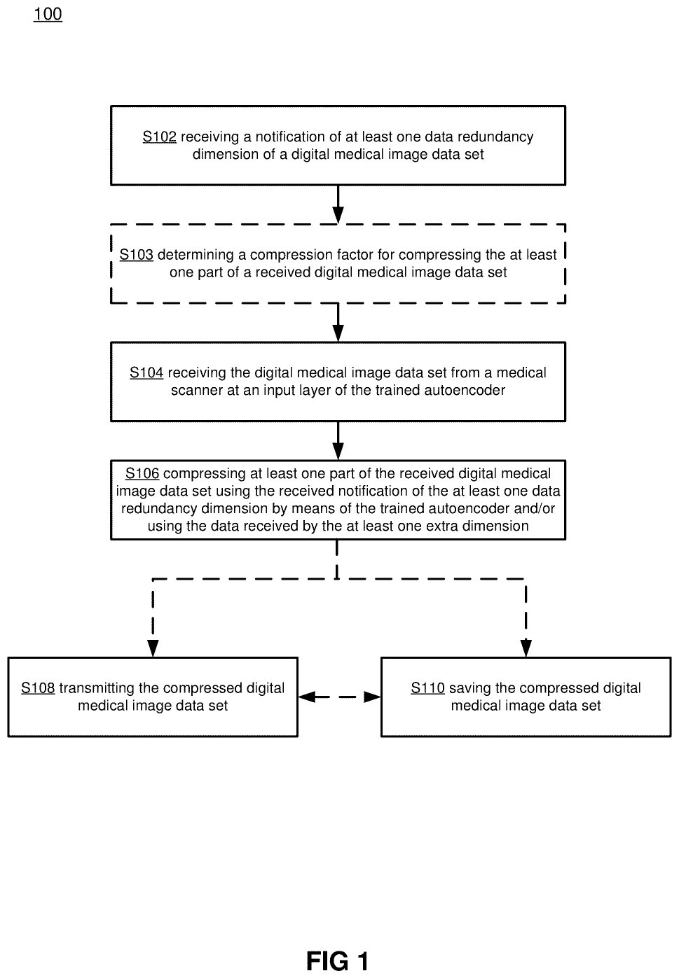

FIG. 1 shows an example schematic flow chart of a (in particular computer-implemented) method 100 for compressing a digital medical image data set, which was obtained using a medical scanner. The method 100 can be executed in an inference phase of a trained autoencoder.

The method 100 may comprise a step S102 of receiving a notification of at least one data redundancy dimension of a digital medical image data set.

A trained autoencoder can be selected (not shown) in response to the received notification of the at least one data redundancy dimension.

The method 100 may further comprise a step S104 of receiving the digital medical image data set from a medical scanner at an input layer of the trained autoencoder.

The trained autoencoder can comprise at least one extra dimension in the input layer. The trained autoencoder may also optionally comprise at least one extra dimension in an output layer.

The at least one extra dimension (in particular in the input layer) can be designed to receive S102 the notification of at least one data redundancy dimension of the received digital medical image data set. Alternatively, or additionally, the at least one extra dimension (in particular in the input layer, and optionally in the output layer) is provided in response to the received S102 notification of the at least one data redundancy dimension of the received digital medical image data set. Alternatively, or additionally, the at least one extra dimension in the input layer is also designed to receive data with regard to the data redundancy dimension. The data received at the at least one extra dimension in the input layer may comprise at least one part of the received S104 digital medical image data set (selected in particular with regard to the data redundancy dimension).

The method 100 may further comprise a step S106 of compressing at least one part of the received S104 digital medical image data set using the received S102 notification of the at least one data redundancy dimension by means of the trained autoencoder and/or using the data received by the at least one extra dimension.

The method 100 can optionally comprise a step S108 of transmitting the compressed S106 digital medical image data set. Alternatively, or additionally, the method 100 can comprise a step S110 of saving the compressed S106 digital medical image data set.

The method 100 can optionally also comprise a step S103 of determining a compression factor for compressing S106 the at least one part of the received S104 digital medical image data set.

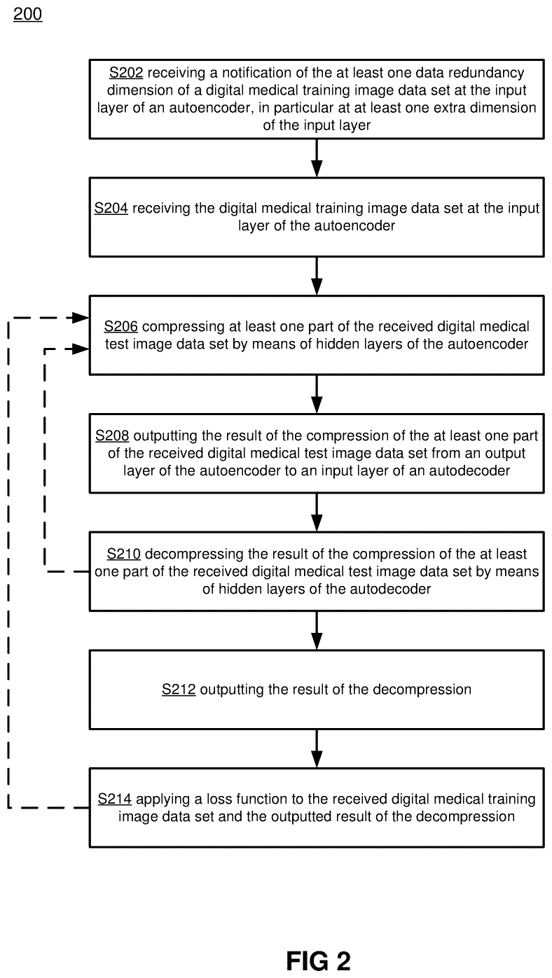

FIG. 2 shows an example schematic flow chart of a training phase 200 of an autoencoder on the data redundancy dimension. The autoencoder can execute the method 100 for compressing a digital medical image data set after the training phase 200 in the inference phase.

The training phase 200 can comprise a step S202 of receiving a notification of the at least one data redundancy dimension of a digital medical training image data set at the input layer of an autoencoder at, for example, at least one extra dimension of the input layer.

The digital medical training image data set can have been obtained using a medical scanner. Alternatively, or additionally, the digital medical training image data set can have been produced synthetically, for example, by means of a generative adversarial network (GAN), by means of a variational autoencoder (VAE) and/or by means of another suitable neural network.

The training phase 200 can further comprise a step S204 of receiving the digital medical training image data set at the input layer of the autoencoder. In particular, step S204 may comprise receiving data with regard to the data redundancy dimension at the at least one extra dimension of the input layer. The data received at the at least one extra dimension in the input layer can comprise at least one part of the received S204 digital medical training image data set (selected in particular with regard to the data redundancy dimension).

The training phase 200 can further comprise a step S206 of compressing at least one part of the received S204 digital medical test image data set by means of hidden layers of the autoencoder. In particular, the compression S206 can be effected using the received S202 notification of the at least one data redundancy dimension of the digital medical training image data set and/or using data received at the at least one extra dimension.

The training phase 200 can further comprise a step S208 of outputting the result of the compression S208 of the at least one part of the received S204 digital medical test image data set from an output layer of the autoencoder to an input layer of an autodecoder. Outputting S208 the result can comprise sending a notification of the data redundancy dimension used for the compression S206 to the output layer of the autoencoder, which is transmitted to the input layer of the autodecoder, for example, concatenated with the compressed digital medical test image data set.

The training phase 200 can further comprise a step S210 of decompressing the result of the compression S208 of the at least one part of the received S204 digital medical test image data set by means of hidden layers of the autodecoder, in particular based on the transmitted S208 notification of the data redundancy dimension used for the compression S206.

The training phase 200 can further comprise the steps S212 and S214 of outputting the result of the decompression S210 or applying a loss function to the received S204 digital medical training image data set and the outputted S212 result of the decompression S210.

At least the steps of compressing S206 by the autoencoder, outputting S208 by the autoencoder to the autodecoder and of decompressing S210 by the autodecoder can be repeated, in order to optimize the applied S214 loss function.

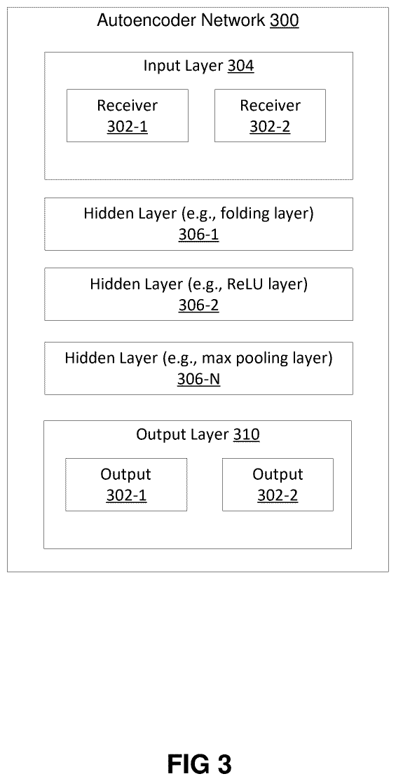

FIG. 3 shows a schematic exemplary embodiment of an (in particular trained) autoencoder network 300 for compressing a digital medical image data set, which was obtained using a medical scanner.

The autoencoder network 300 may comprise an input layer 304. The input layer 304 may comprise a section 302-1, which may be configured for conventional receiving of the digital medical image data set. The input layer 304 may optionally further comprise at least one extra dimension 302-2. The at least one extra dimension 302-2 can be designed to receive a notification of at least one data redundancy dimension of the received digital medical image data set. Alternatively, or additionally, the at least one extra dimension 302-2 can be provided in response to a received notification of at least one data redundancy dimension of the received digital medical image data set. Alternatively, or additionally, the at least one extra dimension 302-2 in the input layer 304 can also be designed to receive data with regard to the data redundancy dimension. The data received at the at least one extra dimension 302-2 in the input layer 304 can comprise at least one part of the received digital medical image data set (selected in particular with regard to the data redundancy dimension).

The autoencoder network 300 may comprise a plurality of hidden layers 306. The hidden layers 306 may be configured to compress the digital medical image data set, and may be referred to as a compressor or compressing processor. The hidden layers 306 can in particular comprise folding layers, ReLU layers, and max pooling layers (not shown in detail). The autoencoder network 300 may further comprise an output layer 310. The output layer 310 may comprise a section 308-1, which may be configured for conventional outputting of the compressed digital medical image data set. The output layer 310 in the exemplary embodiment in FIG. 3 can further comprise at least one extra dimension 308-2. The at least one extra dimension 308-2 can be provided in response to a received notification of at least one data redundancy dimension of the received digital medical image data set (in particular at the input layer 304, specifically at the at least one extra dimension 302-2). The extra dimension 308-2 can provide a notification with regard to the at least one data redundancy dimension used for later decoding by an (e.g., physically separate) decoder, for example, by means of concatenating the notification with the compressed digital medical image data set.

The trained autoencoder network 300 may be configured to compress at least one part of the received digital medical image data set using a notification of the at least one data redundancy dimension.

The autoencoder network 300 may be configured to execute the method 100 in an inference phase. Alternatively, or additionally, the autoencoder network can be trained by means of the training phase 200. In an exemplary embodiment, the autoencoder network 300 includes processing circuitry that is configured to perform one or more functions and/or operations of the autoencoder network 300. Additionally, or alternatively, one or more components of the autoencoder network 300 includes processing circuitry that is configured to perform one or more respective functions and/or operations of the component(s).

A system (not shown) for compressing a digital medical image data set, which was obtained using a medical scanner, can comprise a scanner interface to at least one medical scanner and at least one autoencoder network 300. In particular, the system can comprise a plurality of medical scanners (e.g., with different modalities) and/or a plurality of autoencoder networks 300 (e.g., each trained for a predetermined application).

The disclosure (in particular comprising the method 100, the training phase 200, the autoencoder network 300, and/or the system) can in particular be a method for compressing MRI data by means of autoencoder networks.

The principle of the autoencoder and related network architectures is applied to medical (e.g., MRI) imaging by means of the technology according to the disclosure. Possible applications arise in the compact storage of large data sets (e.g., Human Connectome Project (HCP) diffusion and fMRI data sets) and/or in the rapid transmission of raw data via a network or the internet. The latter is interesting, e.g., for applications in which reconstruction tasks are outsourced to a cloud or to computer clusters.

According to the disclosure, a data redundancy in medical (e.g., MRI) imaging is provided. The data redundancy is utilized, for example, by using the dimension in which a redundancy is expected as at least one extra dimension in the input layer (and optionally in the output layer). In conventional, non-medical 2D image compression this extra dimension would be spanned by the RGB channels.

For medical time-series scans, such as fMRI or contrast imaging, the time dimension, for example, is the extra dimension.

In conventional medical 2D imaging, the extra dimension can comprise the channel dimension or the layer dimension. Depending on the expected redundancy, it can be useful to use the k-space, the image space, or a hybrid space as input data. Other trajectories outside 2D imaging are also conceivable. For spiral or radial imaging, for example, the time sequence of an ADC scan, e.g., also a spatial dimension of the input layer of the autoencoder, the coil dimension in the other spatial dimension, and the time dimension, can be arranged (and/or sorted) as an extra dimension.

Alternatively, or additionally, there can be a connection both to the scan time and to the (e.g., actual) k-space position. E.g., instead of arranging k-space line next to k-space line side by side as usual, the chronological adjacency can be encoded in the extra dimension. For example, the coil dimension can be used as a second spatial dimension.

For predetermined (and/or specific) imaging protocols, which are always executed with the same imaging parameters (e.g., HCP-DTI protocols or fMRI protocols), dedicated autoencoders can be trained. Extremely good compression results can be obtained through specific tuning to this data.

For clinical 2D/3D imaging, for which the matrix size, layer number, and coil number often vary, different approaches (also combinable) can be selected. In a first exemplary embodiment, multiple autoencoder networks are trained for different dimensions or applications.

In a second exemplary embodiment (combinable with the first exemplary embodiment), one or more autoencoder networks are trained with high dimension sizes and zero-filling of the input data. The zero-filling can be performed in each dimension, e.g., as zero-filling of a 384×384 image matrix for a 512×512 autoencoder input layer and/or as zero-filling of a scan with twenty (20) layers in an input layer, which was trained for forty (40) layers in the extra dimension.

In a third exemplary embodiment (combinable with the first and/or second exemplary embodiment), sliding window encoding is affected. If a layer of a 20-layer scan is to be encoded with an autoencoder whose input layer only has three (3) layers in the extra dimension, the two adjacent layers can also be used. The extra dimension of the output layer of the decoder can then be 1. Consequently, the data redundancy is nevertheless considered with the adjacent layers.

In a fourth exemplary embodiment (combinable with the first, second, and/or third exemplary embodiment), the Hermitian k-space symmetry is exploited. The four quadrants of the k-space can be moved to the same orientation by means of mirroring and complex conjugation and used as the extra dimension.

Alternatively, or additionally, a fourth dimension can be spanned in the input layer, and/or different dimensions (e.g., for coil and layer) can be joined together (and/or concatenated) in an extra dimension.

In another type of application (that can be combined with each preceding exemplary embodiment) only parts of the input data can be encoded, while other parts are saved and/or transmitted without being compressed. Depending on the application, for example only the k-space center, only the k-space periphery, or alternating different parts of the k-space can be encoded.

Other potential applications arise in the coil compression (here the selected channel number of the decoder can or must be lower than that of the encoder if necessary) and/or in the transmission of detector data for the CT or PET imaging, in particular if the transmission bandwidth is limited.

The technology according to the disclosure applies an autoencoder network architecture for data compression exploiting redundancy in digital medical image data sets (e.g., comprising MRI data). Advantageously, this facilitates a lower storage space requirement and/or faster data transmission.

An example application using the technology according to the disclosure is the compression of k-space data, which is achieved after the image has been reconstructed from the complex image data using Fourier transformation. This can be particularly useful in the event that reconstructed large data sets need to be saved in a compressed state, e.g., HCP scans. The HCP scans comprise in particular fMRI time series. In the case of fMRI time series, it can be useful to use the complex image data directly without first transforming it back into the k-space.

For applications in which data has to be transmitted quickly from the scanner to a server for reconstruction, only k-space data is usually available. Image data cannot usually be produced yet, as this only happens in the course of the reconstruction. The k-space data or the hybrid space data can be used directly here (in particular after FT along the readout direction; this FT can occur in most instances, as this direction is usually available with Nyquist sampling) for compression by means of the technology according to the disclosure.

An alternative or additional application for compression of the hybrid space data by means of the technology according to the disclosure arises for so-called readout oversampling: a field of view that is more than twice as high is usually scanned in the readout direction (as described in particular at https://mriquestions.com/frequency-wrap-around.html, the content of which is incorporated by reference herein). In most cases, in which no parts of the body are in the readout direction in this direction (e.g., axial head imaging), these areas receive almost no signal and thus usually have good compressibility. Compression without a redundancy dimension is also possible here, if the data is transposed into the hybrid space by FT before compression along the readout direction.

Different data redundancy dimensions depending on the application (and/or imaging protocol), in particular for MRI, are listed as examples in the following table.

Example data redundancy dimension per
application, in particular for MRI

| | Dimension with high data |
|---|---|
| Application | redundancy expected |
| Time-series scans (e.g., fMRI and/or | Time dimension |
| dynamic contrast-enhanced) | |
| Conventional 2D scan | Layer dimension |
| Multi-contrast scan (e.g., T1/T2/T2* | Contrast dimension |
| mapping and/or DIXON imaging) | |
| All scans with multiple coil channels | Coil dimension |

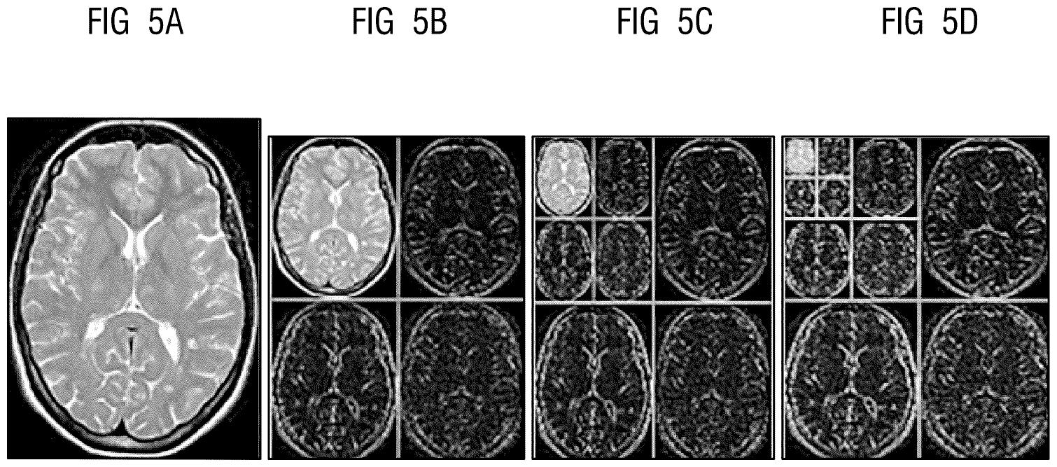

FIGS. 5A, 5B, 5C, and 5D show an example of a WT of a head image reproduced by Ali Alijamaat et al., “Multiple sclerosis identification in brain MRI images using wavelet convolutional neural networks”, Int. J. Imaging Syst. Technol., Vol 31, No 2, pages 778-785 (2021), the content of which is incorporated by reference herein. FIG. 5A shows the original image, and FIG. 5B, 5C, and 5D show different WT results or spectral decompositions of the image. The black (and/or homogeneous) areas after the WT in FIG. 5B, 5C, and 5D each indicate good compressibility.

In another exemplary embodiment, an FT may comprise a cardiac MRI time series in the time direction. Only Fourier coefficients with low frequency are found in places with little changes in the image; the remainder is approximately zero. Thus, data compression by means of the technology according to the disclosure is advantageous after FT in the time direction.

Insofar as not already explicitly described, individual embodiment variants or their individual aspects and features, which are described in reference to the drawings, can be combined or replaced with one another, without limiting or expanding the scope of the described disclosure, if such a combination or such a replacement is useful and for the purpose of the present disclosure. Advantages that are described in reference to a specific embodiment variant of the present disclosure or in reference to a specific figure are, where applicable, also advantages of other embodiment variants of the present disclosure. Gender-neutral language is used throughout this text.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for compressing a digital medical image data set, which was obtained using a medical scanner, comprising:
receiving a notification of at least one data redundancy dimension of a digital medical image data set to be received;
receiving, at an input layer of a trained autoencoder, the digital medical image data set from the medical scanner, wherein the trained autoencoder comprises at least one extra dimension in an input layer, the at least one extra dimension in the input layer being configured to receive data with regard to the at least one data redundancy dimension, wherein the data received at the at least one extra dimension comprises at least one part of the received digital medical image data set selected with regard to the at least one data redundancy dimension;
compressing, using the trained autoencoder, at least one part of the received digital medical image data set based on: the received notification of the at least one data redundancy dimension, and/or data received at the at least one extra dimension, to generate a compressed digital medical image data set; and
providing the compressed digital medical image data set in electronic form as output data.

2. The method as claimed in claim 1, wherein providing the compressed digital medical image data set comprises:
transmitting the compressed digital medical image data set; and/or storing the compressed digital medical image data set.

3. The method as claimed in claim 1, wherein the received notification of the at least one data redundancy dimension is:
information with regard to a type of a data redundancy;
a selection of the trained autoencoder from a number of trained autoencoders, wherein each autoencoder is trained for a predetermined type of data redundancy; and/or
a copy of at least one part of the digital medical image data set.

4. The method as claimed in claim 1, wherein the at least one extra dimension in an output layer of the trained autoencoder is configured to provide autodecoder information with regard to the received notification that has been used in the compression of the at least one part of the received digital medical image data set.

5. The method as claimed in claim 1, further comprising: determining a compression factor for the compression of the at least one part of the received digital medical image data set.

6. The method as claimed in claim 1, wherein the received digital medical image data set comprises at least one of the following data types:
raw data of a scan from the medical scanner;
data in a transformed space;
data in k-space of a magnetic resonance imaging, MRI, scan;
data in the, in particular complex, image space of a scan from the medical scanner; and
data in a hybrid space of an MRI scan.

7. The method as claimed in claim 1, wherein the data redundancy dimension comprises at least one of the following dimensions:
a time dimension, where the digital medical image data set comprises a time-series scan;
a repetition dimension, where the digital medical image data set comprises a number of repetitions;
a contrast dimension, where the digital medical image data set comprises a multi-contrast scan;
a channel dimension, where the digital medical image data set comprises a multi-channel scan:
a layer dimension, where the digital medical image data set comprises a multi-layer scan; and
a coil dimension, where the digital medical image data set comprises a scan with multiple coils.

8. The method as claimed in claim 1, wherein the digital medical image data set is obtained from the medical scanner in accordance with a predetermined imaging protocol.

9. The method as claimed in claim 1, wherein:
the at least one compressed part of the digital medical image data set comprises an area in image space; and/or
the medical scanner comprises a magnetic resonance image (MRI) and the at least one compressed part of the digital medical image data set comprises a k-space center, a k-space periphery, and/or alternating different parts of k-space.

10. The method as claimed in claim 1, wherein the compression of the at least one part of the received digital medical image data set is further based on and/or the received notification of the at least one data redundancy dimension comprises:
a sliding window encoding of adjacent layers within the digital medical image data set and/or adjacent chronological instances of a time-series scan within the digital medical image data set;
a symmetry property of the digital medical image data set, in particular a Hermitian k-space symmetry of a magnetic resonance imaging (MRI) scan; and/or
an oversampling along a readout direction, in particular for a digital medical image data set comprising data in the hybrid space of an MRI scan.

11. The method as claimed in claim 1, wherein the autoencoder is trained on the data redundancy dimension, comprising:
receiving, at the input layer of the autoencoder and at at least one extra dimension of the input layer, a notification of at least one data redundancy dimension of a digital medical training image data set, wherein the digital medical training image data set was obtained using a medical scanner;
receiving, at the input layer of the autoencoder (300), the digital medical training image data set, including receiving data with regard to the data redundancy dimension at the at least one extra dimension of the input layer, wherein the data received at the at least one extra dimension in the input layer comprises at least one part of the received digital medical training image data set selected with regard to the data redundancy dimension;
compressing, by hidden layers of the autoencoder, at least one part of received digital medical test image data set based on the received notification of the at least one data redundancy dimension of the digital medical training image data set and/or data received at the at least one extra dimension;
outputting, by an output layer of the autoencoder to an input layer of an autodecoder, a result of the compression of the at least one part of the received digital medical test image data set;
decompressing, by the hidden layers of the autodecoder, the result of the compression of the at least one part of the received digital medical test image data set; and
outputting the result of the decompression and application of a loss function to the received digital medical training image data set and to the outputted result of the decompression,
wherein at least operations of compressing by the autoencoder, outputting by the autoencoder to the autodecoder, and of decompressing by the autodecoder are repeated, to optimize the applied loss function.

12. The method as claimed in claim 11, further comprising a backpropagation and/or a gradient method for training the autoencoder.

13. The method as claimed in claim 1, wherein the autoencoder is large-dimensioned and/or has an option for omitting input data depending on a type of data redundancy dimension and digital medical training image data set, wherein the autoencoder is trained with a plurality of different types of digital medical training image data sets and data redundancy dimensions.

14. A non-transitory computer-readable storage medium with an executable program stored thereon, wherein, when executed, the program instructs a processor to perform the method of claim 1.

15. A trained autoencoder network for compressing a digital medical image data set, which was obtained using a medical scanner, comprising:
an input layer configured to receive the digital medical image data set, wherein the input layer comprises at least one extra dimension to: receive a notification of at least one data redundancy dimension of the received digital medical image data set, and/or receive data with regard to the data redundancy dimension including at least one part of the received digital medical image data set selected with regard to the data redundancy dimension;
a compressor configured to compress at least one part of the received digital medical image data set using the received notification of the at least one data redundancy dimension and/or using data received at the at least one extra dimension; and
an output layer configured to provide a notification of the data redundancy dimension used for compression.

16. The autoencoder network as claimed in claim 15, wherein the output layer comprises at least one extra dimension provided in response to a received notification of at least one data redundancy dimension of the received digital medical image data set, the at least one extra dimension comprising and/or being configured to provide the notification of the data redundancy dimension used for compression.

17. A system for compressing a digital medical image data set, which was obtained using a medical scanner, comprising:
a scanner interface to at least one medical scanner; and
the autoencoder network according to claim 15.
